(19) United States
(12) Patent Application Publication
(10) Pub. No.: US 2012/0301937 A1
(43) Pub. Date: Nov. 29, 2012

(54) METHODS FOR PRODUCING AND HARVESTING ETHANOL AND APPARATUS FOR PRODUCING AND HARVESTING THE SAME

(75) Inventor: Dan Nilsson, Aalborg (DK)

(73) Assignee: Scale Biofuel, ApS, Aalborg (DK)

(21) Appl. No.: 13/574,366

(22) PCT Filed: Jan. 26, 2011
(86) PCT No.: PCT/IB2011/050347

§ 371 Date: Jul. 20, 2012

(30) Foreign Application Priority Data
Jan. 26, 2010 (DK) 201000059

(51) Int. Cl. C12P 7/06; C12M 1/107; C12M 1/00

(52) U.S. Cl. 435/161; 435/289.1; 435/300.1

(57) ABSTRACT

The invention relates to methods for producing and harvesting ethanol from fermentable sugars derived from sugar crops, starch-containing and lignocellulose-containing materials, and apparatuses for producing and harvesting the same.

### CROSS-REFERENCE TO RELATED APPLICATION

This application claims priority from the following Danish priority application DK 2010 00059 filed on Jan. 26, 2010. That application is incorporated in its entirety by reference herein.

### TECHNICAL FIELD

The invention relates to methods for producing and harvesting ethanol from fermentable sugars derived from sugar crops, starch-containing and lignocellulose-containing materials, and apparatuses for producing and harvesting the same.

### BACKGROUND OF THE INVENTION

The escalating cost of fossil fuels and the increased world demand for such fuels have generated a market shift to the development and use of alternative fuels for energy needs such as transportation, heating, and electricity generation.

The most common alternative fuel for the transportation sector is fuel ethanol. The primary sources of fuel ethanol being produced today are corn (US) and sugarcane (Brazil), but the preferred source of the future is likely cellulosic biomass. Regardless of the source of fermentable sugars for generating fuel ethanol, the capital expenditure (CAPEX) required to building a commercial plant to produce 50 to 100 million gallons of fuel ethanol per year is estimated at $100M-$200M USD. Thus, a large financial hurdle exists, in even the most developed countries, to substantially increase fuel ethanol production due to the CAPEX required to build new production facilities. In lesser developed countries, this CAPEX requirement essentially eliminates the possibility of those countries producing domestic fuel ethanol.

Therefore, low cost methods and apparatuses for producing and harvesting fuel ethanol are highly desirable.

### SUMMARY OF THE INVENTION

In one aspect, the present invention provides a method for producing and harvesting ethanol comprising placing into an enclosed main vessel a fermentation medium comprising fermentable sugars and a fermenting organism capable of fermenting such fermentable sugars into ethanol, fermenting the fermentable sugars with the fermenting organism to produce ethanol, evaporating the ethanol as a gas into a headspace above the fermentation medium within the main vessel, and condensing the gas-phase ethanol into liquid-phase ethanol condensate in a condensing unit, wherein the cooling portion of the condensing unit is located substantially or completely below the ground surface.

In another aspect, the present invention provides an outdoor apparatus for producing and harvesting ethanol comprising an enclosed main vessel comprising a lower portion and an upper portion, wherein the lower portion contains a fermentation medium and the upper portion contains a headspace above the fermentation medium, a condensing unit comprising a cooling portion wherein the cooling portion is located substantially or completely under the surface of the ground, and one or more means for connecting the enclosed main vessel and the condensing unit comprising one or more pipes or tubes capable of transporting gases, liquids, solids or a combination thereof, to and from the enclosed main vessel and the condensing unit.

### BRIEF DESCRIPTION OF THE DRAWINGS

FIG. 1 shows an apparatus of the present invention.

FIG. 2 shows the temperature fluctuations, air flow, and accumulated ethanol condensate produced in a day/night simulation of a method of the present invention.

### DETAILED DESCRIPTION OF THE INVENTION

Disclosed herein is a method for producing and harvesting a fermentation product such as ethanol from fermentable sugars and an apparatus for producing and harvesting the same.

The method of the present invention takes advantage of natural solar energy and the thermal gradient at the ground surface to produce and harvest ethanol utilizing an apparatus wherein the ethanol is partially or completely produced in a main vessel, the ethanol is evaporated as a gas into the enclosed headspace of the main vessel, and the ethanol is condensed into liquid-phase ethanol condensate in a separate condensing unit located substantially or completely below the ground surface.

In the first aspect, the present invention provides a method for producing and harvesting ethanol comprising placing into an enclosed main vessel a fermentation medium comprising fermentable sugars and a fermenting organism capable of fermenting such fermentable sugars into ethanol, fermenting the fermentable sugars with the fermenting organism to produce ethanol, evaporating the ethanol as a gas into a headspace above the fermentation medium within the main vessel, and condensing the gas-phase ethanol into liquid-phase ethanol condensate in a condensing unit, wherein the cooling portion of the condensing unit is located substantially or completely below the ground surface.

In one embodiment, the fermentable sugars and the fermenting organisms are combined prior to placing them into the main vessel. For example, such sugars and organisms can be combined in a mixing tank located proximately or remotely to the main vessel. The mixing tank can be further used as a propagation tank for propagating the fermenting organisms. The mixing tank can be subject to temperature and/or pressure regulation in accordance with the requirements for propagation of the selected fermenting organisms and can further be used as a fermentation vessel for producing ethanol. The liquid material in the mixing tank comprising the fermentable sugars and fermenting organisms can form the fermentation medium that is placed into the main vessel for further fermentation and/or evaporation.

The method, according to the present invention, is carried out in an outdoor apparatus comprising an enclosed main vessel and a condensing unit. The main vessel comprises an upper portion and a lower portion. The lower portion of the main vessel contains fermentation medium and the upper portion of the main vessel contains the headspace above the fermentation medium. The upper portion of the main vessel can be made of clear plastic or other material that allows sunlight or solar energy to enter the main vessel. In one embodiment, the upper portion of the main vessel may further comprise one or more devices, covers, or other shade producing or insulating members to reduce the amount of solar energy entering the main vessel and/or to reduce heat loss from inside the main vessel. The lower portion of the main vessel containing the fermentation medium can be made of any material suitable for containing the liquid medium. The lower portion of the main vessel can be located on the surface of the ground, elevated above the surface of the ground, or located partially or full below the surface of the ground. The height above or depth below the ground surface of the lower portion of the main vessel depends upon the amount of insulation or cooling desired for the fermentation medium contained in the lower portion of the main vessel. The main vessel contains one or more inlets and discharges for introducing and removing solids, liquids and/or gases. The condensing unit of the apparatus is a separate unit from the enclosed main vessel and a substantial portion or the entire portion of the cooling portion of the condensing unit is located underneath the ground surface. The condensing unit can be any structure or apparatus suitable for condensing the gas-phase ethanol into a liquid-phase ethanol condensate. In one embodiment, the ethanol concentration in the liquid-phase ethanol condensate is higher than the concentration of ethanol in the fermentation medium. The cooling portion of the condensing unit comprises one or more conduit and/or loop systems having one or more inlets and discharges and is geothermally cooled. The exact design of the conduit and/or loop systems can be determined by those skilled in the art and is primarily determined by the amount of cooling capacity desired. Secondary considerations for selection of a conduit and/or loop system design include the amount of land available, the ground temperature and composition, the proximity of a sufficiently deep body of water should a lake loop design be desired, and the material and installation cost associated with constructing the system. The appropriate design and amount of cooling achieved by one or more loop systems at various ground temperatures for a particular conduit or loop system can be determined, for example, using ground loop design geothermal software such as provided by Gaia Geothermal, www.gaiageo.com. Geothermally cooled loop systems such as those used in geothermal heating and cooling systems typically are horizontal loop, vertical loop, or lake loop designs. Such system designs can be used or adapted for use in the method of the present invention.

The main vessel and the condensing unit are connected by one or more means for transporting gases, liquids, solids, or a combination thereof, to and from the main vessel and the condensing unit. In one embodiment, the lower chamber comprises at least one discharge for removing all or part of the fermentation medium from the main vessel. The removed fermentation medium may contain solids and can be transported by any suitable means such as a tube or pipe, optionally with the aid of a pump, to a centrifuge where some or all of the solids can be removed from the fermentation medium. Some or all of the solids can be further processed for incorporation into feed such as dry distillers grains (DDGs) for cattle or other livestock feed. All or part of the liquid portion of the fermentation medium, and some or all of the solids, can be transported directly back to the main vessel, or to a mixing tank. In one embodiment, the upper portion of the main vessel comprises at least one inlet for receiving gases such as air, CO₂, or some combination thereof, and at least one discharge for removing the gas-phase ethanol from the upper portion of the main vessel for condensing into a liquid-phase ethanol condensate in the condensing unit. The condensing unit comprises at least one inlet for receiving gas-phase ethanol from the main vessel and one or more means for collecting liquid such as the liquid-phase ethanol condensate. In one embodiment, the condensing unit further comprises at least one discharge in which gases such as air, CO₂ or a combination thereof is returned to the main vessel. The liquid-phase ethanol condensate can optionally be removed from the condensing unit through a discharge in the condensing unit for further ethanol enrichment, such as through further distillation by any means, or storage. Such further distillation or storage can occur proximately and/or remotely to the ethanol production and harvesting main vessel.

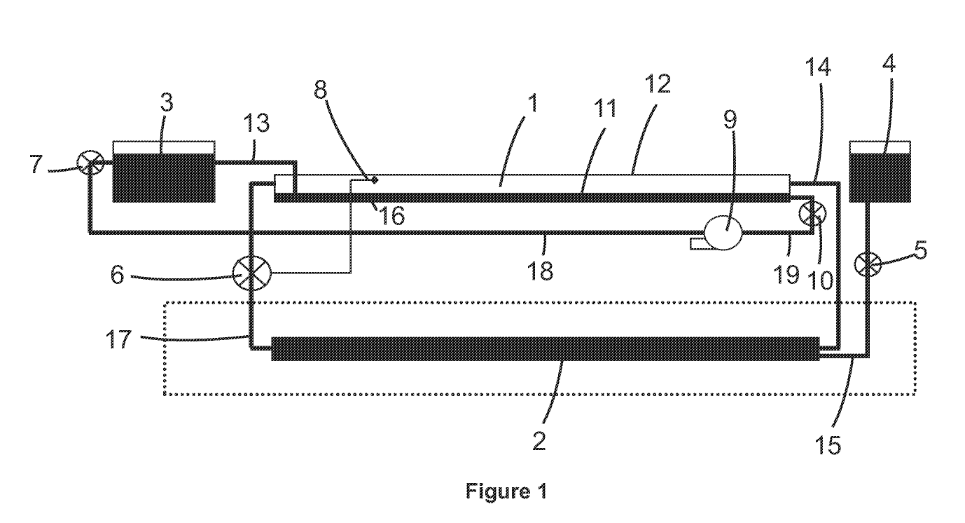

FIG. 1 is a schematic view of one embodiment of the apparatus for producing and harvesting ethanol according to the invention. Main vessel 12 comprises an upper portion or headspace 1 and a lower portion 11 containing the fermentation medium. The apparatus further comprises a condensing unit 2 wherein the cooling portion of the condensing unit is located substantially or completely under the surface of the ground. The main vessel and the condensing unit are connected via one or more means of transporting gas-phase ethanol from the main vessel to the condensing unit such as by pipe section 14. The condensing unit 2 further comprises at least one discharge for transporting gases such as air, CO₂, or mixtures thereof, back to the headspace 1 of the main vessel via a pipe section 17. Such gases may also contain gas-phase ethanol that has not condensed into liquid-phase ethanol condensate in the condensing unit. Gases are removed from condensing unit 2 and returned to the headspace 1 through pipe section 17 optionally with the aid of pump 6. Condensing unit 2 further comprises a discharge for removing or recovering liquid-phase ethanol condensate from the condensing unit. Liquid-phase ethanol condensate can be removed from the condensing unit 2 by pump 5 through pipe section 15 for recovery and/or storage in tank 4. The embodiment further comprises a discharge in the lower portion of the main vessel for removing all or part of the fermentation medium from the main vessel. The removed fermentation medium may contain solids and can be transported via pipe section 19 to a centrifuge 9, optionally with the aid of pump 10, wherein some or all of the solids can be removed from the fermentation medium. Some or all of the solids can be further processed for incorporation into feed such as dry distillers grains (DDGs) for cattle or other livestock feed. All or part of the liquid portion of the fermentation medium can be transported to mixing tank 3 via pipe section 18 with the aid of pump 7. Fermenting organisms, fermentable sugars, and/or other components of the fermentation medium can be prepared or combined in mixing tank 3. Mixing tank 3 is connected to the main vessel by a means for transporting, by gravity feed or optionally with a pump (not shown), all or a portion of the contents of mixing tank 3 to the main vessel 12 such as by pipe section 13. The embodiment may further comprise temperature probe 8 for measuring the temperature of the headspace 1, and temperature probe 16 for measuring the temperature of the fermentation medium in the lower portion 11 of the main vessel.

In one embodiment, the main vessel 12 comprises a lower portion 11 constructed as a pond of about 50 m² to about 5000 m² with an average depth of about 0.1 m to about 0.5 m. The pond can be lined with any suitable material capable of containing the fermentation medium. In another embodiment, the main vessel 12 comprises an upper portion 1 constructed of clear or translucent glass or plastic and measuring about between 0.1 m and about 2 m high, and enclosed with the same or different clear or translucent glass or plastic, such enclosing achieved with a flat, sloped, or domed roof-like structure optionally optimized for the desired amount of solar energy entering the main vessel.

In one embodiment the method of the present invention employs the natural solar energy to elevate the temperature in the headspace of the upper portion of the main vessel. During the daylight hours, the headspace heats up relatively quickly. Not being bound by any particular theory, such temperature increase in the headspace causes an increase in the rate of evaporation of the ethanol in the fermentation medium in the lower portion of the main vessel. In one embodiment, the gas-phase ethanol is forced out of the headspace by pump 6 into the condensing unit 2 located substantially or completely under the ground surface. The cooling portion of the condensing unit is geothermally cooled so the gas-phase ethanol entering the condensing unit is rapidly cooled to form liquid-phase ethanol condensate. As the gases pass into and through the condensing unit the gases are cooled by the cooler ground temperatures causing the gas-phase ethanol to condense into liquid-phase ethanol condensate. The pumping of gases through the apparatus via pump 6 can also in one embodiment be used to aid in regulating the temperature of the headspace and/or the fermentation medium. During the daylight hours the fermentation medium also heats up as a result of the solar energy entering the main vessel. In one embodiment, during daylight hours the fermentation medium does not heat up as quickly nor to as high a temperature as the headspace during the same daylight hours. This differential in temperature between the headspace and the fermentation medium may be advantageous with respect to increasing the evaporation rate of the ethanol in the fermentation medium while maintaining a temperature in the fermentation medium that is suitable for the fermenting organisms to grow and produce ethanol.

In one embodiment, temperature probes 8 and 16 can be used to regulate the temperature of the headspace and the fermentation medium in the main vessel, respectively. Temperature probes 16 and 8 can be connected to a monitoring device capable of regulating certain pumps, valves, or other components related to regulating the temperature of the upper and lower portions of the main vessel. For example, if the headspace of the main vessel is higher than desired, the rate at which pump 6 circulates gases through the apparatus can be increased. Additionally, if it is desired to keep the temperature of the headspace elevated as long as possible, even after sunset, the rate at which pump 6 circulates gases through the apparatus can be decreased. Such increases and decreases in the rate of gas flow can be automatically regulated with one or more devices typically used to monitor temperature changes and regulate mechanical devices within structures such as greenhouses. See, for example, devices such as the Sensaphone monitoring systems provided by Absolute Automation, Casco, Mich., USA. In one embodiment, the device can be programmed such that the airflow changes to a predetermined rate based upon the temperature of the headspace. Further, in order to maintain the temperature of the fermentation medium, the depth of the fermentation medium can be adjusted with the addition or removal of fermentation medium to or from the lower portion of the main vessel. Additional fermentation medium or components of the fermentation medium can be added directly to the main vessel from any source or can be added to the main vessel by removing fermentation medium or components of fermentation medium from mixing tank 3, by gravity feed or with the aid of a pump, and transporting it into the lower portion of the main vessel via pipe section 13. The fermentation medium added to the main vessel can be warmer or cooler than the fermentation medium in the lower portion of the main vessel such that the temperature of the resulting mixture of fermentation medium is higher or lower than the temperature of the fermentation medium in the lower portion of the main vessel just prior to the addition of the fermentation medium or components thereof.

The method of the present invention can be adapted for use in many locations around the world depending primarily upon the average hours and intensity of sunlight (i.e average solar insolation level in kWh/m²/day) and the average temperature of the ground in any particular location. For purposes of the present invention, the apparatus can be located in any suitable location wherein during daylight hours the solar insolation level is sufficient enough to heat the headspace of the main vessel to a temperature at least 5° C. greater than the average temperature of the ground at a depth of 10 feet in the same location.

The amount of solar insolation available in a given location will affect the temperatures of the headspace and the fermentation medium in the method of the present invention. In one embodiment, the apparatus is located in a location wherein the average solar insolation level is at least 3.0 kwh/m²/day and the average ground temperature at a depth of 10 feet is between about 5° C. and about 30° C. In another embodiment, the average solar insolation level is at least 4.0 kwh/m²/day and the average ground temperature at a depth of 10 feet is between about 10° C. and about 30° C.

In one embodiment, the method of the invention comprises maintaining the fermentation medium at a temperature between about 4° C. and about 70° C. In a further embodiment, the method of the invention comprises maintaining the temperature of the fermentation medium at a temperature between about 20° C. and about 70° C., and in a further embodiment the temperature of the fermentation medium is maintained between about 30° C. and about 70° C.

The desired temperature of the fermentation medium depends substantially on the fermenting organism selected. In one embodiment, one or more yeast or bacterial thermophiles are employed in the method of the present invention. In one embodiment, one or more thermophiles such as genetically engineered Geobacillus sps. as described, for example, in RE Cripps et al., Metabolic Engineering 11 (2009) 398-408, are selected for use in the method of the present invention.

The maximum temperature achieved in the headspace of the main vessel depends on the amount of solar energy that enters the vessel during daylight hours and the amount of heat retained in the main vessel during the non-daylight hours. In one embodiment of the present invention, the method comprises heating the headspace of the main vessel during daylight hours to a maximum temperature between about 4° C. and about 85° C. In another embodiment, the headspace of the main vessel is heated to a maximum temperature between about 25° C. and about 70° C. during daylight hours. In a further embodiment, the headspace of the main vessel is heated to a maximum temperature between about 40° C. and about 65° C. during daylight hours.

Due to the rising and setting of the sun, the amount of solar energy entering the main vessel will fluctuate during a 24 hour period. Therefore, in one embodiment, the temperature of the headspace and the temperature of the fermentation medium will fluctuate over a 24 hour period. In an effort to maintain or decrease the natural fluctuation of the temperature of the headspace or the temperature of the fermentation medium during the method of the present invention, one or more temperature regulation methods can be employed. In one embodiment, the depth of the fermentation medium can be adjusted in order to decrease the amount of temperature fluctuation in the fermentation medium during a 24 hour period. In another embodiment, the rate that the gases are pumped through the apparatus can be increased or decreased to decrease the temperature fluctuation in the headspace during a 24 hour period.

Microorganisms such as yeast and some bacteria are capable of fermenting sugars to produce ethanol. Sugars that bacteria and yeast are capable of directly or indirectly converting into ethanol are herein referred to as “fermentable sugars.” For purposes of the present invention, examples of fermentable sugars include, but are not limited to, sucrose, glucose, fructose, xylose, mannose, and galactose, or any saccharide typically containing five or six carbon atoms that can be directly or indirectly fermented into ethanol by certain fermenting organisms. In one embodiment of the method of the present invention, the fermentable sugars are at a concentration of about 10-50% w/v in the fermentation medium.

Fermentable Sugars from Sugar Crops

Certain sugar crops such as sugarcane, sugar beets, and sweet sorghum contain a large amount of fermentable sugars that can be fermented directly or indirectly into ethanol by certain fermenting organisms. For example, at the time of harvest, sugarcane generally contains about 90% sucrose and about 10% combined glucose and fructose. Such sugars are extracted from the sugarcane in the form of sugarcane juice. The juice, syrup, or the molasses produced as a byproduct of the process for producing sugar from sugarcane, can directly or indirectly be fermented into ethanol by certain fermenting organisms such as yeast.

Fermentable Sugars from Starch-Containing Material

Production of fermentation products, such as ethanol, from starch-containing material is well-known in the art. Starch-containing materials for purposed of the present invention include, but are not limited to, corn, wheat, grain sorghum, barley, cassava, and potatoes. Generally two different kinds of processes are used to generate fermentable sugars from starch-containing material. The most commonly used process, often referred to as the “conventional process,” includes liquefaction of gelatinized starch at high temperature using typically a bacterial alpha-amylase, followed by saccharification carried out in the presence of a glucoamylase. Another well-known process, often referred to as a “raw starch hydrolysis” process (RSH) includes saccharifying granular starch below the initial gelatinization temperature typically in the presence of an acid fungal alpha-amylase and a glucoamylase. In both the conventional and raw starch processes, saccharification can be carried out separately or simultaneously with fermentation. According to the present invention, saccharification of gelatinized starch can occur prior to fermentation. Preferably, according to the present invention, if starch-containing material is the source of fermentable sugars for the present invention, the fermentable sugars will be generated utilizing a raw starch hydrolysis process prior to or concurrent with fermentation. The RSH can occur in the same main vessel as the fermentation and evaporation, or the RSH can occur in a separate vessel located proximately or remotely to the fermentation and evaporation main vessel. Hydrolysis of starch-containing materials by either method described above is well known in the art and is described, for example, in WO/2010/022045.

Fermentable Sugars from Lignocellulose-Containing Biomass

In order to obtain fermentable sugars from lignocellulose-containing biomass, the cellulose and hemicellulose components of the biomass must be broken down into fermentable sugars by hydrolysis. Examples of lignocellulose-containing biomass for purposes of the present invention, include but are not limited to corn fiber, rice straw, pine wood, wood chips, bagasse, paper and pulp processing waste, corn stover, corn cobs, hard wood such as poplar and birch, soft wood, cereal straw such as wheat straw, rice straw, switch grass, Miscanthus, rice hulls, municipal solid waste (MSW), industrial organic waste, office paper, or mixtures thereof.

Methods for producing fermentable sugars from lignocellulosic biomass are well known in the art and such methods typically combine one or more processes such as pretreatment and/or acid or enzymatic hydrolysis. Methods for obtaining fermentable sugars from lignocellulose-containing materials are described, for example, in WO/2010/039812. Other suitable sources include lignocellulose-derived sugars by radical chain reaction chemistry such as GAF catalysis of lignocellulosic material by Georgia Alternatives Fuels, LLC, Georgia, U.S.A.

In one embodiment, the fermentable sugars are obtained from one or more sugar crops such as sugarcane, sugar beets, and sweet sorghum. In another embodiment, the fermentable sugars are obtained from starch-containing materials such as corn. In another embodiment, the fermentable sugars are obtained from one or more lignocellulose-containing materials such as switch grass and bagasse. In a further embodiment, the source of fermentable sugars is a concentrated sugar feedstock as from Sweetwater Energy, Inc., Rochester, N.Y., U.S.A.

### Fermenting Organisms

The term “fermenting organism” refers to any organism, including bacterial and fungal organisms, including yeast and filamentous fungi, suitable for producing ethanol. Especially suitable fermenting organisms according to the invention are able to ferment, i.e., convert sugars, such as sucrose, glucose, fructose, maltose, xylose, mannose and/or arabinose, directly or indirectly into ethanol. Examples of fermenting organisms include fungal organisms, such as yeast. Contemplated strains of yeast include strains of the genus Saccharomyces, in particular a strain of Saccharomyces cerevisiae or Saccharomyces uvarum; a strain of Pichia, in particular Pichia stipitis or Pichia pastoris; a strain of the genus Candida, in particular a strain of Candida utilis, Candida arabinofermentans, Candida diddensii, Candida sonorensis, Candida shehatae, Candida tropicalis, Candida digboiensis, Candida thermophila, or Candida boidinii. Other contemplated yeast includes strains of Hansenula, in particular Hansenula polymorpha or Hansenula anomala; strains of Kluyveromyces, in particular Kluyveromyces marxianus or Kluyveromyces fagilis, and strains of Schizosaccharomyces, in particular Schizosaccharomyces pombe.

Contemplated bacterial fermenting organisms include strains of Escherichia, in particular Escherichia coli, strains of Zymomonas, in particular Zymomonas mobilis, strains of Zymobacter, in particular Zymobactor palmae, strains of Klebsiella in particular Klebsiella oxytoca, strains of Leuconostoc, in particular Leuconostoc mesenteroides, strains of Clostridium, in particular Clostridium butyricum, strains of Enterobacter, in particular Enterobacter aerogenes and strains of Thermoanaerobacter, in particular Thermoanaerobacter BG1 L1 (Appl. Microbiol. Biotech. 77: 61-86) and Thermoanarobacter ethanolicus, Thermoanaerobacter thermosaccharolyticum, or Thermoanaerobacter mathranii. Strains of Lactobacillus are also envisioned as are strains of Corynebacterium glutamicum R, Bacillus thermoglucosidaisus, and Geobacillus thermoglucosidasius.

In connection with especially fermentation of lignocellulose derived fermentable sugars, C5 sugar fermenting organisms are contemplated. Most C5 sugar fermenting organisms also ferment C6 sugars. Examples of C5 sugar fermenting organisms include strains of Pichia, such as of the species Pichia stipitis. C5 sugar fermenting bacteria are also known. Also some Saccharomyces cerevisiae strains ferment C5 (and C6) sugars. Examples are genetically modified strains of Saccharomyces spp. that are capable of fermenting C5 sugars include the ones concerned in, e.g., Ho et al., 1998, Applied and Environmental Microbiology, p. 1852-1859 and Karhumaa et al., 2006, Microbial Cell Factories 5:18, and Kuyper et al., 2005, FEMS Yeast Research 5, p. 925-934.

Certain preferred fermenting organisms include Candida thermophila as described by Shin et al., Int J Syst Evol Microbiol, 51: 2167 (2001); a modified Bacillus strain as described in U.S. Pat. No. 7,691,620; one or more Geobacillus strains as described in Tang et al., Biotechnology and Bioengineering, 102: 1377-1386 (2009); Kluyveromyces marxianus as described in Babiker et al., Appl Microbiol Biotechnol (2010) 85:861-7; and the ethanol producing mesophilic and thermophilic organisms described in WO2006/117536, WO2008/038019, WO2008/141174, WO2009/022158, WO2010/052499, and those described by RE Cripps et al., Metabolic Engineering 11 (2009) 398-408.

For purposes of the present invention, the term “thermophile” means a microorganism that grows optimally at temperatures between about 40° C. and about 85° C., yet also includes organisms that can grow or withstand temperatures as low as about 4° C. and as high as about 105° C. According to the method of the invention, selection of the fermenting organism depends primarily on the source of fermentable sugars, the temperature range at which fermentation is carried out, and the level of ethanol tolerance of the fermenting organism. One skilled in the art can readily select a fermenting organism for use in the present invention based on these and other parameters understood by those skilled in the art. According to the method of the present invention, the fermenting organism can be a naturally occurring organism or a genetically modified organism.

The amount of ethanol in the fermentation medium can be regulated by one or more means of concentration or dilution. For example, as ethanol evaporates out of the fermentation medium the concentration of ethanol in the fermentation medium typically decreases. In one embodiment, in order to increase the concentration of ethanol in the fermentation medium, additional fermentable sugars can be added to the main vessel in a continuous or batch fashion at suitable conditions for the fermenting organisms to produce additional ethanol. In another embodiment, in order to decrease the concentration of ethanol, additional fermentation medium without fermentable sugars or with less fermentable sugars can be added. Based on the ethanol tolerance of the fermenting organism selected, one skilled in the art can determine the desired range of ethanol concentrations to be maintained in the fermentation medium.

In one embodiment one or more fermenting organisms are added to the fermentation medium so that the viable fermenting organism, such as yeast, count per ml of fermentation medium is in the range from 10⁵ to 10¹², preferably from 10⁷ to 10¹⁰, especially about 5×10⁷. Commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

As used herein, the phrase “fermentation media” or “fermentation medium” refers to the aqueous environment in which fermentation is carried out and comprises the fermentation substrate, that is, the carbohydrate source that is metabolized by the fermenting organisms to produce the fermentation product, and may include the fermenting organisms. The fermentation medium may further comprise nutrients and growth stimulators for the fermenting organisms. Nutrient and growth stimulators are widely used in the art of fermentation and include nitrogen sources, such as ammonia, vitamins and minerals, or combinations thereof. Following fermentation, the fermentation media or fermentation medium may further comprise the fermentation product such as ethanol.

### EXAMPLES

### Materials and Methods

### Preparation of “Fermentation Medium”

Ethanol produced by yeast fermentation: 4 kg of sugar was dissolved in tap water to a final volume of approximately 12.5 liter with 59 g dry yeast (Turbo Pure, Gert Strand AB, Malmoe, Sweden) and incubated in the fermentation vessel at 28-30° C. for approximately 93 hours. The ethanol concentration in the fermentation medium at the end of 93 hours was approximately 15.7% as determined by HPLC (K. Ohgren et al. Biomass and Bioenergy 30 (2006) 863-869).

The fermentation medium was at a depth of approximately 10 cm in the fermentation vessel. The fermentation vessel dimensions were a height of 40 cm, a length of 40 cm and a width of 32 cm. The vessel was constructed of dark brown polyethylene on the sides and bottom, and the top of the vessel was closed with an 8 mm thick glass plate. The vessel was insulated on the sides and bottom with Rockwool 50 mm stone wool insulation (TUN No.: 16 24 527, Rockwool A/S, Hedehusene, Denmark) with an insulating capacity at 50° C. equal to 44 mW m⁻¹ K⁻¹. Air was circulated through the headspace of the vessel at a rate of approximately 5.6 to 15 m³/hour per m² of exposed surface area of the fermentation medium. In the present example, the exposed surface are of the fermentation medium is 0.128 m², thus the corresponding air flow was approximately 12 to 32 L/min. The air in the headspace above the fermentation medium was lead to the condensing unit via a ⅜″ inner diameter hose. The condensing unit consisted of a plate condenser constructed from 16 aluminum plates contained in a unit being 34 cm×24 cm×4.5 cm (plate condenser) supplied by Auto og Industri køler centret, Aalborg, Denmark, in line with a hose condenser constructed from a ⅜″ plastic hose wrapped around a 10 L plastic bucket filled with room temperature water (hose condenser). The distal end of the ⅜″ inner diameter hose of the hose condenser was connected to a Y shaped plastic connector for gravity separation of the liquid condensate from the air flow. One outlet of the Y shaped plastic connector was connected with a ⅜″ inner diameter hose to a sealed 1 liter Bluecap bottle for collecting the liquid condensate. The remaining outlet of the Y connector was connected with a ⅜″ plastic tube to a diaphragm air pump with a capacity of 33 L/min (B100, Charles Austen Pumps Ltd, Surrey, UK) regulated by a potentiometer regulated frequency converter (Motron FC750, Eltwin A/S, Risskov, Denmark) for returning the air that passed through the condensing unit back to the fermentation vessel. The cooling capacity of the combined plate and hose condensing unit was approximately 18 W at a room temperature of between 18° C. and 22° C.

Six 60 W white light spot lamps (Concentra Spot R63, Osram Gmbh, Munich, Germany) hanging approximately 50 cm above the vessel provided radiant heat to the vessel. Based on the temperature increase in the fermentation medium and the vessel headspace, it was determined that the average solar insolation of the white lights is roughly equivalent to 5.4 kwh/m²/day.

The ethanol concentration in the condensate was measured with an alcohol meter (Alkoholmeter tysk 30 cm, Vinøl Hobby, Frederiksberg, Denmark) calibrated to 0% v/v with tap water and 100% with denatured 93% v/v ethanol. Following each measurement the ethanol condensate was returned to the fermentation vessel.

### Example 1

### Day/Night Simulation

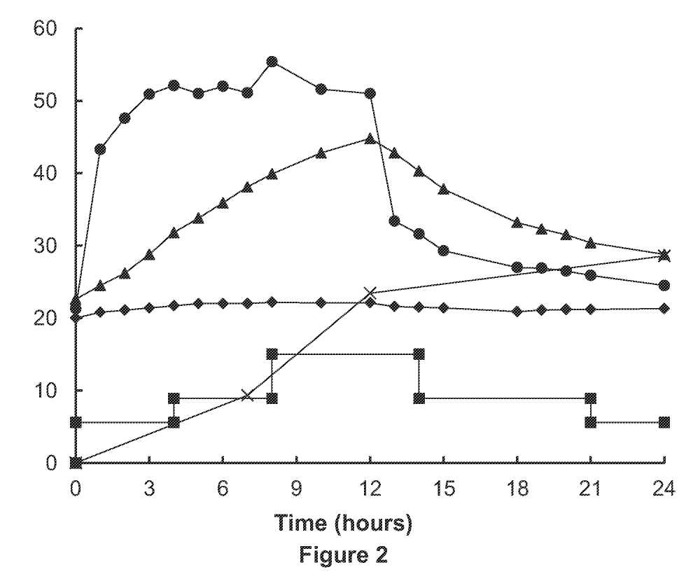

White light spot lamps were turned on for 12 hours and then turned off for 12 hours to simulate a natural day/night cycle. The combined plate and hose condensing unit was assembled as described above. CO₂ was circulated through the headspace and condensing unit at a rate of approximately 5.6, 8.9 or 15 m³/hour per m² exposed surface area of fermentation medium. The exposed surface area of the fermentation medium was 0.128 m². The temperature of the fermentation medium [symbol] , headspace [symbol] , and of the room [symbol] , the air flow [symbol] , the amount of condensate per time period, percent ethanol in the condensate, and the accumulated amount of ethanol condensate per m² exposed fermentation medium [symbol] , were measured over a 24 hour period and are displayed in Table 1 and graphically in FIG. 2. As stated in the materials and method section above, following each measurement of the ethanol condensate, the ethanol condensate was returned to the fermentation vessel. Therefore, the fermentation medium contained a relatively constant ethanol concentration of 15.7% v/v.

TABLE 1

| | Temp ° C. | | | | | | |
|---|---|---|---|---|---|---|---|
| Time | (fermentation | Temp ° C. | Temp ° C. | Air flow | Condensate | Ethanol | Accumulated |
| hours | medium) | (room) | (headspace) | m³/m²h | dl | % v/v | ethanol dl/m² |
| 0 | 22.6 | 20.0 | 21.3 | 5.6 | 0.00 | | 0.0 |
| 1 | 24.5 | 20.8 | 43.3 | 5.6 | | | |
| 2 | 26.2 | 21.1 | 47.6 | 5.6 | | | |
| 3 | 28.8 | 21.4 | 50.9 | 5.6 | | | |
| 4 | 31.8 | 21.7 | 52.1 | 8.9 | | | |
| 5 | 33.8 | 22.0 | 51.0 | 8.9 | | | |
| 6 | 35.9 | 22.0 | 52.0 | 8.9 | | | |
| 7 | 38.1 | 22.0 | 51.1 | 8.9 | 3.32 | 36 | 9.3 |
| 8 | 39.9 | 22.2 | 55.4 | 15.0 | | | |
| 10 | 42.8 | 22.1 | 51.6 | 15.0 | | | |
| 12 | 44.8 | 22.1 | 51.0 | 15.0 | 4.40 | 41 | 23.4 |
| 13 | 42.8 | 21.6 | 33.4 | 15.0 | | | |
| 14 | 40.3 | 21.5 | 31.6 | 8.9 | | | |
| 15 | 37.8 | 21.4 | 29.3 | 8.9 | | | |
| 18 | 33.2 | 20.9 | 27.0 | 8.9 | | | |
| 19 | 32.3 | 21.1 | 26.9 | 8.9 | | | |
| 20 | 31.5 | 21.2 | 26.5 | 8.9 | | | |
| 21 | 30.4 | 21.2 | 25.9 | 5.6 | | | |
| 24 | 28.8 | 21.3 | 24.5 | 5.6 | 2.06 | 32 | 28.6 |

### Example 2

In reference to FIG. 1, an apparatus according to the invention can consist of a 4 m³ mixing vessel 3 located in or under an enclosed or otherwise shade-providing structure, a 5 m wide and 20 m long and 0.4 m high main vessel 12 containing fermentation medium of 0.1 m depth in the lower portion 11 of the main vessel. The lower portion 11 of the main vessel can be located at a depth of approximately 0.1-0.2 m into the ground. The lower portion 11 of the main vessel can be partially or completely insulated by insulation material suitable for obtaining a higher heat transfer to the fermentation medium in the main vessel from the solar energy entering the main vessel than if the main vessel was not insulated from the ground. The apparatus can further comprise a horizontal geothermal condenser approximately 10 feet down into the ground and the condenser can be constructed such that at an air flow of 1500 m³ per hour through the main vessel the condensing unit can condense 60° C. gas-phase ethanol to form liquid-phase ethanol condensate at 30° C. or lower. The apparatus can be located in Malaysia, New Delhi, India, Hong Kong, China, or Miami Fla. and can produce approximately 10,000 to 100,000 gallons per year. The fermenting organism can be Geobacillus sp. Strain TM242 and the fermentable sugars can comprise sugarcane juice, molasses, syrup, or other condensed sugar source at a concentration of 10-50% w/v fermentation medium.

What is claimed is:

1. A method for producing and harvesting ethanol comprising:
placing into an enclosed main vessel a fermentation medium comprising fermentable sugars and a fermenting organism capable of fermenting such fermentable sugars into ethanol;
fermenting the fermentable sugars with the fermenting organism to produce ethanol;
evaporating the ethanol as a gas into a headspace above the fermentation medium within the main vessel; and
condensing the gas-phase ethanol into liquid-phase ethanol condensate in a condensing unit, wherein the cooling portion of the condensing unit is located substantially or completely below the ground surface.

2. The method of claim 1, wherein the fermentable sugars and the fermenting organism are combined prior to placing them into the main vessel.

3. The method of claim 1, further comprising heating the headspace of the main vessel with solar energy.

4. The method of claim 1, further comprising cooling the cooling portion of the condensing unit geothermally.

5. The method of claim 1, wherein the fermentable sugars are derived from one or more sources selected sugar crops, starch-containing material, lignocellulose-containing materials, or any combination thereof.

6. The method of claim 1, wherein the fermenting organism is a thermophile.

7. The method of claim 1, wherein the fermenting organism is a genetically modified organism.

8. The method of claim 1, wherein the ethanol condensate has an ethanol concentration that is higher than the ethanol concentration in the fermentation medium.

9. The method of claim 1, wherein the ethanol condensate is recovered from the condensing unit.

10. The method of claim 9, wherein the ethanol condensate is subject to distillation.

11. An outdoor apparatus for producing and harvesting ethanol comprising:
an enclosed main vessel comprising a lower portion and an upper portion, wherein the lower portion contains a fermentation medium and the upper portion contains a headspace above the fermentation medium;
a condensing unit comprising a cooling portion wherein the cooling portion is located substantially or completely under the surface of the ground; and
one or more means for connecting the enclosed main vessel and the condensing unit comprising one or more pipes or tubes capable of transporting gases, liquids, solids or a combination thereof, to and/or from the enclosed main vessel and the condensing unit.

12. The apparatus of claim 11, wherein the upper portion of the main vessel comprises at least one inlet for receiving gasses and at least one discharge for removing gas-phase ethanol from the upper portion of the main vessel for condensing in the condensing unit.

13. The apparatus of claim 11, wherein the condensing unit comprises at least one inlet for receiving gas-phase ethanol from the main vessel.

14. The apparatus of claim 11, wherein the condensing unit comprises at least one means for collecting liquid.

15. The apparatus of claim 11, wherein the condensing unit comprises at least one discharge for removing gasses from the condensing unit.

16. The apparatus of claim 15, wherein the removed gasses are returned to the main vessel through one or more pipes or tubes.

17. The apparatus of claim 11, wherein a pump moves gases through the main vessel and the condensing unit.

18. The apparatus of claim 11, wherein the lower chamber comprises at least one discharge for removing all or part of the fermentation medium with or without solids.

19. The apparatus of claim 11, wherein the apparatus further comprises a centrifuge.

20. The apparatus of claim 11, wherein the apparatus further comprises one or more pumps capable of pumping gases, liquids, solids, or combinations thereof.

21. The apparatus of claim 11, wherein the apparatus further comprises a mixing tank.

22. The apparatus of claim 11, wherein the apparatus further comprises a storage tank.
